(19) United States
(12) Patent Application Publication
(10) Pub. No.: US 2004/0014730 A1
(43) Pub. Date: Jan. 22, 2004

(54) Formulations and methods of reducing toxicity of anti-infective agents

(76) Inventor: Frederick Hausheer, Boerne, TX (US)

Correspondence Address: Thomas J. Dodd, BioNumerik Pharmaceuticals, Inc., Suite 1250, 8122 Datapoint Drive, San Antonio, TX, 78229, US

(21) Appl. No.: 10/192,377

(22) Filed: Jul. 10, 2002

(51) Int. Cl. A61K 31/70; A61K 31/185; A61K 31/66

(52) U.S. Cl. 514/127; 514/553

(57) ABSTRACT

This invention provides for pharmaceutical formulations of compounds that are useful as protective agents when administered to patients also receiving anti-infective drugs, such as antimicrobials, antifungals, or antivirals. The invention also includes methods of reducing the toxicity of various anti-infective agents by administering an effective amount of the protective agent to a patient receiving one or more anti-infective agents. The compounds that are useful as protective agents have either a sulfhydryl moiety or are reducible disulfides.

### FIELD OF THE INVENTION

This invention relates to pharmaceutical formulations of anti-infective drugs and a detoxifying agent. The detoxifying agent is a compound that has one or more sulfhydryl moieties, or a reducible disulfide, and serves to reduce of eliminate the toxic side effects of the anti-infective drug with which it is formulated or administered. The invention also relates to methods of reducing the toxicity of various anti-infective agents by administering to a patient an effective amount of the detoxifying agent before, simultaneously with and/or after administration of the anti-infective agent.

### BACKGROUND OF THE INVENTION

The discovery and development of modern anti-infective and oncology agents has been perhaps the most significant advance in 20^{th} century medicine. From the discovery of penicillin forward, anti-infectives have served to both cure previously life-threatening diseases as well as provide better quality of life by stemming the course of and reducing the spread of other infections.

The treatment of cancer has been advanced by the knowledge derived from research into cellular mechanisms that describe the pathogenesis and pathophysiology of human cancer; this information combined with new knowledge of the biochemical and biological interactions of drugs with molecular targets that underlie the propagation and survival of cancer cells has led to new drug treatments, including novel drugs possessed of both anti-infective and antineoplastic properties.

For purposes of this invention, antibiotic agents are classified as antimicrobial, antifungal or antiviral, based upon the type of organisms they control. Antimicrobial agents are employed to combat diseases caused by infection from microbes (commonly referred to as bacterial infections); antifungal agents are employed mainly to combat fungal infections, and may also be used as antimicrobials; antiviral agents are intended for administration to combat diseases associated with viral infections. Some of these agents, particularly the macrolides and antifolates also are possessed of antineoplastic and other medicinal properties, and may be useful in the treatment of certain cancers, as well as other diseases.

### Antimicrobial Agents

These agents generally fall into one of several classes of drug. The most commonly recognized are the penicillins, cephalosporins, aminoglycosides, tetracyclines, sulfonamides, quinolones, antituberculosis agents, macrolides, other beta-lactam antibiotics, urinary anti-infectives, and others.

Side effects from undesirable toxicity of antimicrobial agents vary widely from class-to-class. Table 1 below illustrates the reported undesirable effects of each class of antimicrobial drug.

TABLE 1

| Class of Antimicrobial Drug | Toxicities |
|---|---|
| Penicillins | 1,2,3,4,6 |
| Cephalosporins | 1,2,3,4,6,8 |
| Aminoglycosides | 1,2,3,5,6 |
| Sulfonamides | 1,2,3,4,6,8 |
| Tetracyclines | 2,3,4,6,8,9,10 |
| Quinolones | 1,2,3,6 |
| Antiruberculosis Agents | 1,2,3,4,5,6,7,8,9 |
| Macrolides | 2,3,4,6,7,8 |
| Urinary Anti-infectives | 2,3,4,6,7,8 |
| Other -lactams | 1,2,3,4,6,8 |
| Antifolates | all |
| Miscellaneous Antimicrobials | all |

Toxicity type-
1. Nephrotoxicity
2. Peripheral Neuropathy and Neurotoxicity
3. Sensitivity Reactions (Local Reactions up to Anaphylaxis)
4. Hepatotoxicity
5. Ototoxicity
6. GI Toxicity (Nausea, Vomiting, Diarrhea, etc.)
7. Cardiopulmonary Toxicity
8. Hematologic Toxicity (anemia, leukopenia, granulocytopenia, thrombocytopenia, neutropenia, etc.)
9. Musculoskeletal Toxicity
10. Other (identified by individual drug)

Severity and frequency of toxicity ranges from rare to frequent and in some cases, requires emergency treatment and/or cessation of therapy. Though most antimicrobials, especially the orally administered agents, are considered very safe for normal usage, adverse effects can negatively impact on treatment success and quality of life.

### Antifungals

Antifungal agents range from topical preparations, which are often sold as over-the-counter drugs for conditions such as candidal yeast infections and athlete's foot, to prescription only medications, to hospital administered IV formulations used in treating serious fungal infections, and fungal diseases such as blastomycosis, coccidiomycosis, aspergillosis, histoplasmosis and others.

Most adverse effects of antifungal drugs (also referred to as antifungal antibiotics) are mild, with the exception of the potent antifungal agent Amphotericin B. Adverse effects of Amphotericin include potentially severe and dose-limiting nephrotoxicity, sensitivity reactions, cardiopulmonary toxicity (including potentially lethal cardiac events), severe nausea and diarrhea as well as other GI effects, anemia, musculoskeletal pain, and others.

### Antivirals

Antiviral agents have been recently developed against certain pathogens such as herpes simplex virus, influenza A, cytomegalovirus, hepatitis C, hantavirus, HIV, and a few others. Many antiviral agents resemble the antineoplastic purine and pyrimidine nucleosides in chemical structure, and exhibit many of the same adverse effects (though of lesser severity in general) as these drugs.

### Pharmaceutical Chemistry of Dimesna, Mesna and Derivatives

Mesna (sodium 2-mercaptoethene sulfonate) and dimesna (disodium 2,2′-dithiobis ethane sulfonate) are known therapeutic compounds that have heretofore demonstrated a wide variety of therapeutic uses. Both mesna and dimesna have been shown to be effective protective agents against certain specific types of toxicity associated with the administration of cytotoxic drugs used to treat patients for various types of cancer.

In particular, mesna has been used with some success in mitigating the toxic effects of cytotoxic agents such as ifosfamide, oxazaphosphorine, melphalane, cyclophosphamide, trofosfamide, sulfosfamide, chlorambucil, busulfan, triethylene thiophosphamide, triaziquone, and others, as disclosed in U.S. Pat. No. 4,220,660, issued Sep. 2, 1980.

The near absence of toxicity of dimesna further underscores the usefulness of this compound, as large doses that may be needed can be given to a patient without increasing the risk of adverse effects from the protective agent itself.

Further, pharmacological profiles of each compound indicate that, if proper conditions are maintained, mesna and dimesna do not prematurely inactivate primary therapeutic drugs to a significant degree. Thus, neither compound will significantly reduce activity of the chemotherapeutic agent, and in many cases, act to potentiate the effect of the main drug on targeted cancer cells.

Chemically, dimesna is a dimer of mesna, with the optimum conditions for oxidation occurring in the slightly basic (pH ⁻7.3), oxygen rich environment found in blood plasma. In mildly acidic, low oxygen conditions, in the presence of a reducing agent such as glutathione reductase, conditions prevalent in the kidneys, the primary constituent is mesna.

Mesna acts as a protective agent for a number of cytotoxic agents by substituting a nontoxic sulfhydryl moiety for a toxic hydroxy (or aquo) moiety. This action is particularly evidenced in the coadministration of mesna and oxazaphosphorine, and in the administration of dimesna along with cisplatin or carboplatin and other agents, such as paclitaxel.

Mesna and dimesna, as well as some analogues of these compounds, have excellent toxicity profiles in mammalian species. In fact, dimesna has been administered intravenously to mice and dogs in doses higher than the accepted oral LD₅₀ for common table salt (3750 mg/kg), with no adverse effects. Dimesna has also been administered to humans in doses exceeding 25 g/m², with no adverse effects.

Mesna, and other analogues with free thiol moieties, constitute the more physiologically active form of the two types of compounds described in this specification. These compounds manifest their activity by providing free thiol moieties for terminal substitution at locations where a terminal leaving group of appropriate configuration is located.

Dimesna and other disulfides are reduced intracellularly by glutathione reductase, a ubiquitous enzyme, thereby generating high concentrations of intracellular free thiols. These free thiols act to scavenge the free radicals and other nucleophilic compounds often responsible for causing cell damage.

This profile is especially significant in explaining the success of dimesna in controlling and mitigating the toxic effects of platinum complex, taxanes and other antitumor drugs. The mechanism for action in the case of cisplatin (cis-diammine dichloro platinum) is explained in U.S. Pat. Nos. 5,789,000 and 5,919,816, which are incorporated herein by reference, as well as in other United States and international patents.

Mesna, dimesna, and analogues of these compounds have been the subject of several prior pharmaceutical uses described in the literature and in prior patents, both in the United States and around the world. In addition to the cytotoxic agent protection uses, one or more of these compounds have proven effective, in vitro, against a multiplicity of biological targets, and have been effective, in vivo, in the treatment of sickle cell disease, radiation exposure, chemical agent exposure, and other uses.

Mesna, dimesna, and analogues thereof are synthesized from commonly available starting materials, using acceptable routes well-known in the art. One such method involves the two-step, single pot synthetic process for making dimesna and like compounds of the following formula:

R₁—S—R₂;

wherein:

R₁ is hydrogen, X-lower alkyl, or X-lower alkyl-R₃;

R₂ is lower alkyl-R₄;

R₃ and R₄ are each individually SO₃M or PO₃M₂;

X is absent or X is sulfur; and

M is an alkali metal.

The process essentially involves a two step single pot synthetic process that results in the conversion of an alkenyl sulfonate salt or acid to the desired formula I compound. The process in the case of mesna is a single step process that converts the alkenyl sulfonate salt to mesna or a mesna derivative by reacting with an alkali metal sulfide or with hydrogen sulfide.

If the desired end product is dimesna or a dimesna analogue, a two-step single pot process is involved. Step 1 is as described above. Step 2 of the process is performed in the same reaction vessel as Step 1 without the need to purify or isolate the mesna formed during that step. Step 2 includes the introduction of oxygen gas into the vessel, along with an increase in pressure and temperature above ambient values, at least 20 pounds per square inch (psi) and at least 60° C. Dimesna or a derivative thereof is formed in essentially quantitative yield.

Other processes, well-known and documented in the prior art, may be employed to make either mesna or dimesna, or derivatives and analogues thereof.

### SUMMARY OF THE INVENTION

This invention relates to the use of Dimesna and analogues and derivatives thereof (hereinafter referred to as “The protective compounds”) to reduce the toxicity of antimicrobial, antifungal or antiviral agents administered to patients as treatment for a bacterial infection, or a fungal infection, or as antiviral therapy. The protective compounds are of the following general formula:

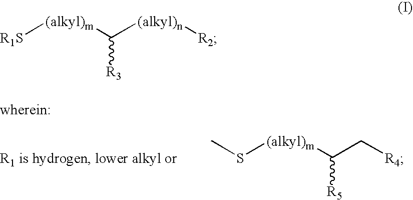

R₂ and R₄ are each individually SO₃⁻M⁺, PO₃²⁻M₂²⁺, or PO₂S²⁻M₂²⁺;

R₃ and R₅ are each individually hydrogen, hydroxy or sulfhydryl;

m and n are individually 0, 1, 2, 3 or 4, with the proviso that if m or n is 0, then R₃ is hydrogen; and

M is hydrogen or an alkali metal ion; or

a pharmaceutically acceptable salt thereof.

Given the pharmacokinetics, as well as the physical, chemical, and biochemical protective properties of the protective compounds, and the proven usefulness of the protective compounds with several structurally different antineoplastic agents, the formula I compounds will be effective in reducing the toxicity of drugs which have similar toxic metabolites and/or mechanisms of action. Particularly, the protective compounds will be useful with nearly all of the drugs listed above, whether the combination of the drug and the protective agent is administered concurrently or separately, and regardless of the route of administration to the patient.

As will be described below, the preferred methods of administration include both the co-administration of the protective compound and the desired agent or agents, as well as separate administration thereof. The preferred route of administration of the antimicrobial, antifungal or antiviral drug will be the most useful and practical route, in most cases by mouth, in a few through intravenous injection or infusion, while the administration of the formula I compound can be either oral or parenteral, irrespective of the method of delivery of the antineoplastic drug. Preferred doses of each agent and a protective compound of formula I are also set forth below.

Accordingly, it is a principal object of this invention to provide for a novel method of treating patients suffering from infectious diseases, by administering an effective amount of i) an antimicrobial agent; and ii) a formula I protective compound as described herein.

Another object of this invention is to provide for a method of reducing the undesirable toxicity, in vivo, of an antimicrobial, antifungal or antiviral agent.

Another object of this invention is to provide for improved and safer methods of treating patients with infectious diseases.

Other objects will become apparent upon a reading of the following description.

### DESCRIPTION OF THE PREFERRED EMBODIMENTS

The preferred embodiments herein described are not intended to be exhaustive or to limit the scope of the invention to the precise forms disclosed. They are chosen and described to best explain the principles of the invention and its application and practical use to allow others skilled in the art to comprehend its teachings.

This invention provides for formulations of compounds having the following formula I in combination with one or more antimicrobial, antifungal or antiviral agents:

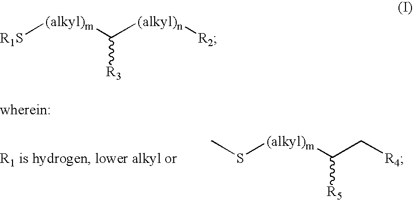

R₂ and R₄ are each individually SO₃⁻M⁺, PO₃²⁻M₂²⁺, or PO₂S²⁻M₂²⁺;

R₃ and R₅ are each individually hydrogen, hydroxy or sulfhydryl;

m and n are individually 0, 1, 2, 3 or 4, with the proviso that if m or n is 0, then R₃ is hydrogen; and

M is hydrogen or an alkali metal ion; or

a pharmaceutically acceptable salt thereof.

As defined above, “antimicrobial agent” means a compound that is administered to a patient as a therapeutic agent for the purpose of treating an infectious disease. Antimicrobial agents with which the formula I compounds may be formulated for use include the following:

Antimicrobial agents—Penicillins, Cephalosporins, Quinolones, Sulfonamides, Antituberculosis Agents, macrolides, other beta-lactam antibiotics, urinary anti-infectives, and others.

It should be noted that the above classifications include antineoplastic agents that may be classified in two or more categories, with some agents actually being classifiable in three or even four of the above categories. The listings provided above are for guidance only, and should not be construed as limiting or all-inclusive. In particular, the macrolides, of which epothilones are a subclass, are possessed of significant antineoplastic activity, and are seen as promising agents for use in the treatment of certain cancers.

The formula I compounds are useful in reducing the common, major toxicities of a large number of anti-infective drugs. The formula I compounds will also no doubt prove useful in reducing the toxicity of other drugs as well, particularly those drugs which generate similar toxic species. In general, the formula I compounds are postulated to be useful in reducing the toxicity of any anti-infective agent which includes one or more hydroxy, aquo, aziridinium, or other moieties which are substitutable by a strong nucleophile, in vivo.

Formulations of the anti-infective agent and the formula I compounds are one of the preferred embodiments of this invention. In this embodiment, the anti-infective agent and the formula I compound may be combined in a single solution, suspension, or other dosable form and packaged for later delivery to the patient.

A second embodiment of this invention relates to the formulation of the formula I compound with a suitable solvent (parenteral formulations) or as a pure drug or formulated with a carrier (oral formulations). The anti-infective agent is packaged in a separate formulation, distinct from the formula I formulation, with the two formulations adapted to be reconstituted and delivered to the patient at the same time as the anti-infective agent. Administration of an effective amount of the formula I compound in all embodiments serves to reduce the undesired toxicity of the anti-infective agent.

A third embodiment of the invention relates to the administration of the formula I compound separately from the anti-infective agent. Delivery of the formula I compound is effected prior to the administration of the anti-infective agent, to reduce the undesired toxicity of the anti-infective agent.

A fourth embodiment of the invention relates to the delivery of the formula I compound after the administration of the anti-infective agent, to reduce the toxicity of the anti-infective agent.

A fifth embodiment of the invention relates to the intermittent administration of the formula I compounds after administration of the anti-infective agent. This route of delivery may be combined with any of the other schedules set forth for the initial administration with the formula I compound.

In all embodiments, the term “effective amount” is understood as a medical art term, that is, the dose schedule and route of administration of the drug that gives the best therapeutic value and convenience to the patient. With regard to anti-infective drugs, and the reduction of toxicity by administration the formula I compounds, an “effective amount” (also referred to as a “detoxifying amount”) of formula I compound is defined as the amount of drug which reduces the clinical manifestation of toxic side effects of the anti-infective drug. In most cases, the range of the effective amount is estimated by the physician using the dose schedule, pharmacokinetic properties, and the patient's weight and body surface area, and adjusting the dose and timing such that the peak concentrations of the protective agent and the peak concentration of the toxic species of the anti-infective agent have the greatest amount of overlap.

Thus, the timing of administration of the formula I compound with respect to the timing of the administration of the anti-infective agent will vary according to the dose, schedule, route of administration, and individual pharmacokinetics of the anti-infective agent being used. The most desired dose ratios, timing, and total amounts of drugs administered, will depend upon the type of anti-infective agent being administered, the toxicities associated with that agent, the overall condition of the patient and the susceptibility of the patient to the anti-infective drug's side effects, the efficacy of the formula I compound with respect to detoxification of the anti-infective agent, and other factors.

The administration of an effective amount of a formula I compound reduces the toxicity of the anti-infective agent(s). The dosage and timing of administration of the formula I compounds is always designed to maximize patient safety throughout the course of the therapeutic regimen. The effectiveness of the formula I compound in achieving the objectives of toxicity reduction and patient safety will depend to some extent on the dosing schedules, and some typical schedules and dosage ratios are described for each anti-infective agent with which the formula I compounds may be administered.

### Co-Formulation of Anti-Infective Agents and Formula I Compounds

The first embodiment of the invention involves the combination of the anti-infective agent and the formula I compound in the same pharmaceutical formulation. The main advantage to a co-formulation of the anti-infective agent and the formula I compound is the ease and convenience of reconstitution by the pharmacist and the nurse, and the ease of administration to the patient. Disadvantages include the potential for premature reactions of the formula I compound and the anti-infective agent that could result in premature inactivation of the anti-infective agent, and failure to achieve toxicity reduction due to different drug cycle times in the body. If the possibility of premature inactivation of the anti-infective agent is a concern, then the two compounds should be formulated separately for administration to the patient.

A typical example of safeguards used to prevent premature reaction of the formulation components is taken from the prior art that involves the combination of cisplatin and Dimesna. In order to prevent the removal of the chlorine groups in favor of the disulfide or sulfhydryl moieties of the formula I compound, the formulation is spiked with additional chloride ions, such as are found in a 0.9% sodium chloride solution. Other examples of safeguards to protect the integrity of the formulation will be apparent to those skilled in the art.

A co-formulation of the formula I compound and the anti-infective agent can take on any of several forms, dependent upon the intended delivery route of the formulation. For purposes of this invention, parenteral, topical and oral formulations will be described.

In a typical parenteral formulation, the two compounds must be dissolved or suspended in a suitable solvent delivery vehicle. Pharmaceutically acceptable solvents are well-known in the art, and by ascertaining the solubility of the formula I compound and the anti-infective drug in various pharmaceutically acceptable solvents, a formulation expert can determine the maximum concentration of both compounds in a preferred formulation. One or more co-solvents can be used if necessary to ensure complete dissolution of the compounds if the desired form of delivery is a solution. Excipients may be added to the solution or suspension to provide for pharmaceutical elegance of the formulation.

The most preferred solvent in many formulations, due to its relative lack of toxicity and ease of delivery, is water. Since the solubility of most formula I compounds is at least 300 mg/mL, the water solubility of the anti-infective agent will determine the usefulness of water as the primary solvent. If the desired dosage of the anti-infective agent and the formula I compound can be fully dissolved in water, such as in the case of many anti-infective agents administered as salts of the free base, then water will be the preferred solvent. As stated above, any chances of premature reaction of the formulation ingredients must be safeguarded against. If delivery of a suspension is preferred, the solubility of the compounds in the solvent(s) important, but not as critical as the solubility when delivering a solution.

In the event an oral formulation is desired, a suitable carrier is necessary. Preferred forms of oral delivery vehicles include filled capsules, pills, caplets, oral solutions or suspensions, tablets, and other common oral dosage forms. Filled capsules may contain either a solution or a suspension of the formula I compound with or without the anti-infective agent. The above disclosure regarding solubility and choice of solvents in parenteral formulations applies also to the oral formulations.

In the event of topical formulations, the preferred forms include lotions, creams, solutions, suspensions, or other forms which can be applied topically. For purposes of this invention, topical formulations also includes those formulations specifically engineered for administration directly into a patient's eye or ear as well.

The preferred dosage of many anti-infective agents is a variable, and is based upon the type of organism involved, other drugs included in the therapeutic regimen, height of the patient, weight of the patient, age of the patient, and in some cases, the gender of the patient. Since the efficacy of the formula I compounds has some dependence on the amounts of both compounds delivered, the preferred formulations are described as weight-to-weight ratios of the formula I compound and the antineoplastic agent. Preferred solvents are also described for each formulation.

TABLE 2

| | | w/w Ratio |
|---|---|---|
| Drug Class | Route | (drug:formula I) |
| Penicillins | Oral or parenteral | 1:5-1:1000 |
| Cephalosporins | Oral | 1:5-1:1000 |
| Antituberculosis Agents | Oral | 1:5-1:1000 |
| Beta-lactams | Oral or parenteral | 1:5-1:1000 |
| Sulfonamides | Oral | 1:5-1:1000 |
| Quinolones | Oral or parenteral | 1:5-1:1000 |
| Antifolates | Oral or Parenteral | 1:3-1:5000 |
| Macrolides | Oral or parenteral | 1:5-1:10,000 |
| Aminoglycosides | Parenteral or oral | 1:5-1:1000 |
| Urinary Anti-infectives | Oral | 1:5-1:1000 |
| Amphotericin | Parenteral | 1:5-1:1000 |
| Azoles | Oral or parenteral | 1:5-1:1000 |
| Pyrimidines | Oral | 1:5-1:1000 |
| Other Anti-fungals | Oral or parenteral | 1:5-1:1000 |
| Antiretrovirals | Parenteral or oral | 1:5-1:1000 |
| Nucleosides and | Parenteral or oral | 1:5-1:1000 |
| Nucleotides | | |
| Other Antivirals | Parenteral or oral | 1:5-1:1000 |

Table 1 depicts the preferred range for dose ratios of the formula I compound with a variety of anti-infective agents. The most preferred dosage ratio will vary depending upon a number of factors in each case, with the principal objective being the safety of the patient.

Steps are also taken to preserve the integrity of the formulation, to prevent the premature reaction of the formula I compound with the anti-infective agent. If there is a danger of the compounds reacting in the formulation, regardless of precautions taken, then they are formulated separately for administration.

Structural formulas for many of the above classes are well known. As examples, the structures of Amphotericin B, and the epothilone macrolide agents are depicted below.

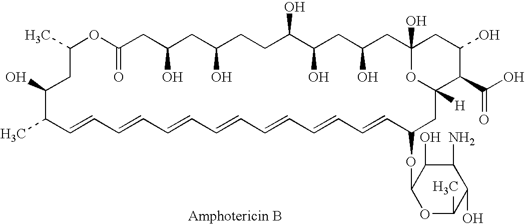

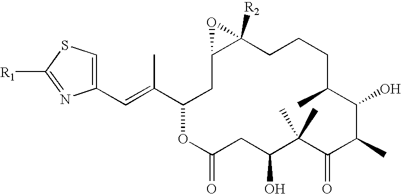

| R₁ | R₂ | Compound |
|---|---|---|
| CH₃ | H | Epo A |
| CH₃ | CH₃ | Epo B |
| CH₂OH | H | Epo E |

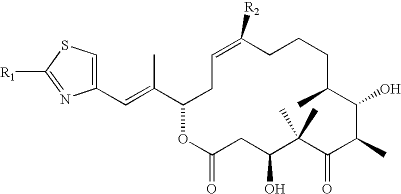

| R₁ | R₂ | Compound |
|---|---|---|
| CH₃ | H | Epo C |
| CH₃ | CH₃ | Epo D |

### Formulations of the Formula I Compounds

The formula I compounds may be formulated for administration apart from the administration of the anti-infective agent. The preferred solvent for making parenteral formulations of the formula I compounds is water. Oral formulations also use water as the preferred primary solvent, if any solvent is used.

The final desired form determines the concentration of the formula I compound in any given parenteral formulation. If the final form is a solution, the upper limit of the concentration of the formula I compound is its maximum solubility in the solvent or solvents selected. If the final form is a suspension, the concentration may be higher.

For oral dosage forms, the total amount of formula I compound present in the dose is preferably an amount that will allow a recommended dose to be conveniently administered. The primary factor in determining the amount of formula I compound contained in oral doses is the required size of the delivery vehicle.

All parenteral and oral formulations of the formula I compounds are designed to be administered to a patient according to the methods taught by this invention. General examples of parenteral and oral formulations of the formula I compounds are depicted below. The most preferred formula I compounds are Dimesna, the disphosphonate analogue of Dimesna (dimephos), the heterodimer of Mesna, where R² is sulfonate, R⁴ is phosphonate (mesnaphos), S-methyl Mesna, and those analogues where one or both of R³ and R⁵ are hydroxy and m and n are at least 1 (hydroxymesna). All of these most preferred compounds have a water solubility of at least 200 mg/mL, with the hydroxy derivatives having the greatest water solubility.

Formulations of the formula I compounds may also include pharmaceutically acceptable excipients, carriers and/or diluents. The composition and amount of each additional material in the formulation will depend upon the desired route of delivery, speed of administration, the timing of drug delivery after administration of the formulation, final desired concentration, and other factors. One preferred excipient that will be included in many formulations is a pH adjustment compound, which is typically either a pharmaceutically acceptable acid or base.

### Use of the Formula I Compounds to Reduce Toxicity of Anti-Infective Agents

This invention also relates to the use of a formula I compound to reduce the undesirable toxic side effects of many anti-infective drugs. Most of the undesired toxic effects of the anti-infective agents are described above.

There are several varying mechanisms, described above, by which most anti-infective agents exert both the desired effects on the target organism, as well as the undesirable toxic effects on normal healthy cells. Administration of a formula I compound in conjunction with one of these anti-infective agents serves to reduce, and in some cases eliminate the toxic side effects associated with the anti-infective agent. Also, due to the pharmacologic properties of the formula I compounds, the reduction of undesired toxicity is not accompanied by a similar fall-off in activity of the anti-infective agent against targeted organisms.

To ensure maximum effect, the formula I compound should be administered such that a suitable concentration of the formula I compound is present in the body to react with the anti-infective agent and metabolites thereof. Preferred timing of the dosage of the formula I compound will depend upon the pharmacologic properties of the particular anti-infective agent, generally from about one minute prior to the administration of the anti-infective agent to about one hour prior to such administration. The most preferred initial route of administration of the formula I compound at this time is by a single IV push, which is administered between fifteen and thirty minutes prior to the administration of the anti-infective agent.

The preferred ratios of administration are depicted in Table 1 above. These ratios are applicable for all routes of initial administration of the formula I compound and the anti-infective agent, whether the two are administered simultaneously or staggered, and whether the two are administered in the same or separate formulations.

In the cases where the reduction of toxicity mechanism of action is known or postulated, the formula I compounds are postulated to reduce the undesired side effect toxicity of the anti-infective agent by displacing terminal leaving groups thereon. Particularly susceptible leaving groups include those groups subject to displacement by a moderate to strong nucleophile, in the formula I compounds represented by the sulfhydryl (and to a lesser extent, the disulfide) moiety.

The leaving groups in many instances are hydroxy moieties, aquo moieties, aziridinium ions, and other displaceable free radical type moieties. In many cases, the anti-infective agent metabolite that contains these moieties has little or no antineoplastic activity, but manifests undesired toxic side effects to healthy cells. Displacement of the terminal leaving group in such cases by the sulfhydryl or disulfide of the formula I compound generates a non-toxic thioether moiety, one that is rapidly eliminated from the body.

Another embodiment involves the administration of the formula I compounds at intermittent times after the administration of the anti-infective agent. This type of administration is seen to be particularly effective when the anti-infective agent has a prolonged and/or multiphasic half-life. Since the formula I compounds are eliminated rapidly from the body (t_{1/2}<90 minutes), administration of the formula I compound at predetermined intervals following administration of the anti-infective agent can provide long term protection against later occurring side effects. This prolonged protection can be extremely beneficial in the case of anti-infective agents that remain in the body in appreciable quantities for extended periods (e.g. t_{1/2}>24 hours).

Combination therapy, where two or more anti-infective agents are administered simultaneously or near simultaneously, presents special considerations in administration of the formula I compounds. Due to the extremely low toxicity of the formula I compounds, large doses, often exceeding 30 grams or more, can be given to the patient as a single dose. Reduction of side effects of combination anti-infective agents is postulated to be dependent upon the pharmacokinetics of the individual agents, and the electrophilic affinity of the displaceable leaving groups of each agent.

The most critical factor of delivering an effective amount of formula I compound to the patient is the maximization of the peak plasma concentration of the protective agent with the peak concentration of the toxic species of the antineoplastic agent. The curve is actually a time versus concentration graph that depicts the concentration of active drug in the bloodstream as a function of time. A typical example of a preferred concentration curve, where the peak concentrations of the formula I compound and the toxic species of the anti-infective agent overlap, is illustrated below as Table 3. An example of a non-preferred peak concentration curve is depicted as Table 4.

TABLE 3

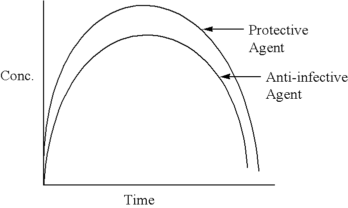

TABLE 4

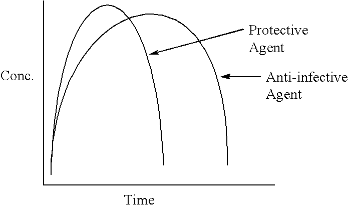

Typical time/concentration curves have been generated for each of the commercial and many other anti-infective drugs so that estimates of the effective amount and timing of administration of each agent can be ascertained. This research enables physicians to determine better courses of therapy for individual patients. In combination therapy in particular, it is often desirable to select agents that have different time/concentration curves, to ensure that the maximum effect of each agent is achieved, and to correspondingly reduce the risk of cumulative drug toxicity. The accepted therapeutic value of a drug in the infectious disease field is referred to as the area under the curve (AUC), which refers to the space occupied beneath the time/concentration curve. More area present beneath this curve is equated with greater therapeutic value from a pharmacokinetic standpoint.

The objective in administering the formula I compounds to the patient is to match as closely as possible the peak concentrations of the toxic species of the anti-infective agent(s) and the formula I compounds. By closely matching peak concentrations of the anti-infective agent and the formula I compound, maximum detoxification can be attained. Since the pharmacokinetics of commercially available anti-infective agents are known or constitute predictable values, the physician can tailor the timing and dosage of the formula I compound to achieve the optimal result.

Dose ratio is also an important factor to evaluate in administering an effective amount of the formula I compound. The dose ratios illustrated above in Table 2 are intended to present guidelines to the practitioner, with actual dose ratios and dose amounts set on a case-by-case basis as the patient's treatment progresses.

Individual treatment regimens, while initially following the prescribed dosing timing and amounts, are often adjusted by to achieve the greatest therapeutic results with concomitantly low risk due to toxic side effects of the anti-infective agent. In many cases, dose reduction of the anti-infective agent can be avoided, with a change in timing and/or an increase in dosage of the protective agent. By allowing the patient to continue to receive high therapeutic doses of the anti-infective agent, the probability of successful treatment is increased.

### SPECIFIC EXAMPLES

The following examples illustrate selected modes for carrying out the claimed invention and are not to be construed as limiting the specification and claims in any way.

### Example 1

### Preparation of 2,2′-Dithio-bis-ethane Sulfonate

2,2′-Dithio-bis-ethane sulfonate is prepared by oxidizing 2-mercapto ethane sulfonate in water with equimolar amount of iodine as previously reported by Lamaire and Reiger (Lamaire and Reiger, J. Org. Chem., 26, 1330-1, 1961).

### Example 2

### Stability of 2,2′-Dithio-bis-ethane Sulfonate

The stability of 2,2′-dithio-bis-ethane sulfonate at room temperature was determined at pH ranges of 1.5 to 9.0. 2,2′-Dithio-bis-ethane sulfonate, as produced by the method described above, was found to be very stable in the pH range of 1.5-9.0.

The following experiment was performed to determine the stability of 2,2′-dithio-bis-ethane sulfonate in acidic and basic aqueous media. In a typical experiment, 50 mg of 2,2′-dithio-bis-ethane sulfonate (as produced by using the above described method) was dissolved in one ml of water and the pH was adjusted to 1.5, 2.0, 3.0, 4.0, 5.0 and 6.0 by adding 1 N hydrochloric acid in water or the pH was adjusted to 8.0 and 9.0 by adding 1 N sodium hydroxide in water. The reaction mixture was stirred for 24 hours at room temperature, the water was removed at reduced pressure, dissolved in spectral grade D₂O, and the proton NMR spectrum was recorded. One peak corresponding to the starting material was observed on the NMR spectra; no additional peaks were observed.

The stability of 2,2′-dithio-bis-ethane sulfonate at pH 1.5 was further studied by heating the reaction mixture to 100 degrees Celsius for 10 minutes. No change in the proton spectrum was observed by heating the 2,2′-dithio-bis-ethane sulfonate (pH 1.5). These data indicate that 2,2′-dithio-bis-ethane sulfonate is stable in aqueous solutions at pH values from 1.5 to 9.0.

### Example 3

### Sterile Solution Containing Amphotericin B and Disodium 2,2′-Dithio-bis-ethane Sulfonate

This example is designed to detail one method to produce a sterile solution containing Amphotericin B and disodium 2,2′-dithio-bis-ethane sulfonate. For the purpose of this example, “approximately” is defined to include a range of plus minus 1%.

Step 1. Sodium phosphates are dissolved in sterile, injectable water to a final concentration of 0.5% by weight of water. A suitable amount of sodium deoxycholate is added. The final pH of the injectable solution is in the range of approximately 2.0 to 6.0.

Step 2. One part by weight of pure Amphotericin B is added to the mixture of Step 1 The Amphotericin B is allowed to completely dissolve by agitation (1500-2500 rpm) at room temperature. A suitable amount of cholesteryl sulfate is added to complex the amphotericin B.

Step 3. 15 parts by weight of disodium 2,2′-dithio-bis-ethane sulfonate (as produced above in Example 1) is added the mixture of Step 2. This mixture is agitated until complete dissolution occurs.

Step 4. The solution of Step 3 is sterilized via filtration through a sterile 0.22 micron filter.

Step 5. The sterile solution of Step 4 is stored in sterile injection vials wherein each vial contains approximately 5 mg of Amphotericin B and 15 g of 2,2′-dithio-bis-ethane sulfonate in the final solution.

### Example 4

### Administration of Epothilone and Disodium 2,2′-Dithio-bis-ethane Sulfonate

Patients with cancer are treated by IV administration of between 0.1 mg/m² to 100 mg/m² of an epothilone (any of the epothilones currently in clinical trials) with the exact dosage amount and timing determined by the clinical trial protocol and by the attending physician. Between 60 minutes prior to 60 minutes following the initiation of the epothilone administration, the patient is also given, by IV administration, between 1.0 g/m₂ to 50 g/m₂ of a formula I compound, preferably disodium 2,2′-dithiobis ethane sulfonate. Administration of the formula I compound is between 50 to 10,000 times greater by weight than the amount of administered epothilone.

The processes for making formulations of the formula I compounds and other anti-infective agents are similar to those described above. The synthetic process for making the formula I compounds, particularly Dimesna, are outlined in U.S. Pat. No. 5,808,140, which is incorporated herein by reference. Methods for using the Formula I compounds with Platinum complex antineoplastic agents are outlined in one or more of the co-pending parent applications and issued patents.

It is noteworthy that, while mechanisms of action of both anti-infective agents and the toxicity reduction features of the formula I compounds have been postulated, the disclosure of these purported mechanisms is not intended to be binding upon the inventors as indicative of the underlying reasons which explain the usefulness of the formula I compounds. As with most anti-infective agents, where the exact mechanism(s) of action is not a certainty, the mechanism(s) of protection by which the formula I compounds reduce the toxicity of the anti-infective agents is not as yet fully understood. However, the above disclosures concerning physiological mechanisms of action constitute the best information known at this time.

What is claimed is:

1. A pharmaceutical formulation comprising a solution or suspension of i) an effective amount of a first drug which comprises an antimicrobial agent or an antifungal agent or an antiviral agent; and ii) a detoxifying amount of a compound of the formula: 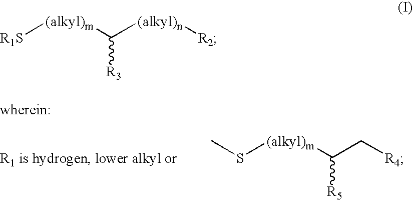
R₂ and R₄ are each individually SO₃⁻M⁺, PO₃²⁻M₂²⁺, or PO₂S²⁻M₂²⁺;
R₃ and R₅ are each individually hydrogen, hydroxy or sulfhydryl;
m and n are individually 0, 1, 2, 3 or 4, with the proviso that if m or n is 0, then R₃ is hydrogen; and
M is hydrogen or an alkali metal ion; or
a pharmaceutically acceptable salt thereof.

2. The pharmaceutical formulation of claim 1 wherein said first drug is an antimicrobial agent.

3. The pharmaceutical formulation of claim 1 wherein said first drug is an antifungal agent.

4. The pharmaceutical formulation of claim 1 wherein said first drug is an antiviral agent.

5. The pharmaceutical formulation of claim 2 wherein said antimicrobial agent is a penicillin.

6. The pharmaceutical formulation of claim 2 wherein said antimicrobial agent is a cephalosporin.

7. The pharmaceutical formulation of claim 2 wherein said antimicrobial agent is a beta-lactam antibiotic.

8. The pharmaceutical formulation of claim 2 wherein said antimicrobial agent is a sulfonamide.

9. The pharmaceutical formulation of claim 2 wherein said antimicrobial agent is a quinolone.

10. The pharmaceutical formulation of claim 2 wherein said antimicrobial agent is a macrolide.

11. The pharmaceutical formulation of claim 10 wherein said antimicrobial agent is an epothilone.

12. The pharmaceutical formulation of claim 2 wherein said antimicrobial agent is an aminoglycoside.

13. The pharmaceutical formulation of claim 2 wherein said antimicrobial agent is an antituberculosis agent.

14. The pharmaceutical formulation of claim 2 wherein said antimicrobial agent is a urinary anti-infective agent.

15. The pharmaceutical formulation of claim 3 wherein said antifungal agent is Amphotericin.

16. The pharmaceutical formulation of claim 3 wherein said antifungal agent is a pyrimidine derivative.

17. The pharmaceutical formulation of claim 3 wherein said antifungal agent is a diazole or triazole-containing compound.

18. The pharmaceutical formulation of claim 4 wherein said antiviral agent is a purine nucleoside or nucleotide.

19. The pharmaceutical formulation of claim 4 wherein said antiviral agent is a pyrimidine nucleoside or nucleotide.

20. The pharmaceutical formulation of claim 4 wherein said antiviral agent is an antiretroviral agent.

21. A method of reducing the toxicity of a first drug consisting of an antimicrobial agent, or an antifungal agent, or an antiviral agent administered to a patient as therapy for an infectious disease, said method comprising administering to said patient an effective amount of said antimicrobial, antifungal or antiviral agent, and a toxicity reducing amount of a compound of the formula: 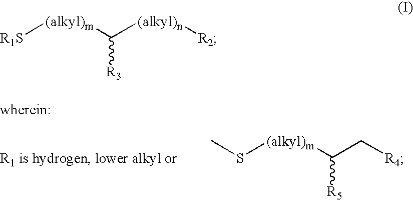
R₂ and R₄ are each individually SO₃⁻M⁺, PO₃²⁻M₂²⁺, or PO₂S²⁻M₂²⁺;
R₃ and R₅ are each individually hydrogen, hydroxy or sulfhydryl;
m and n are individually 0, 1, 2, 3 or 4, with the proviso that if m or n is 0, then R₃ is hydrogen; and
M is hydrogen or an alkali metal ion; or
a pharmaceutically acceptable salt thereof.

22. The method of claim 21 wherein said formula I compound is administered to said patient at a time from five minutes prior to sixty minutes prior to administration of the antimicrobial, antifungal or antiviral agent.

23. The method of claim 21 wherein said formula I compound is administered to said patient at a time from fifteen minutes prior to thirty minutes prior to administration of the antimicrobial, antifungal or antiviral agent.

24. The method of claim 21 wherein said formula I compound is administered to said patient simultaneously with the antimicrobial, antifungal or antiviral agent.

25. The method of claim 22 wherein said formula I compound is administered to said patient intravenously.

26. The method of claim 23 wherein said formula I compound is administered to said patient orally.

27. The method of claim 24 wherein said antimicrobial, antifungal or antiviral agent is administered parenterally.

28. The method of claim 21 wherein said antimicrobial, antifungal or antiviral agent is administered parenterally.

29. The method of claim 24 wherein said antimicrobial, antifungal or antiviral agent is administered orally.

30. The method of claim 21 wherein said first drug is an antimicrobial agent.

31. The method of claim 21 wherein said first drug is an antifungal agent.

32. The method of claim 21 wherein said first drug is an antiviral agent.

33. The method of claim 30 wherein said antimicrobial agent is a penicillin.

34. The method of claim 30 wherein said antimicrobial agent is a cephalosporin.

35. The method of claim 30 wherein said antimicrobial agent is a macrolide.

36. The method of claim 35 wherein said macrolide is an epothilone.

37. The method of claim 30 wherein said antimicrobial agent is a sulfonamide.

37. The method of claim 30 wherein said antimicrobial agent is a quinolone.

38. The method of claim 30 wherein said antimicrobial agent is an aminoglycoside.

39. The method of claim 30 wherein said antimicrobial agent is an antituberculosis agent.

40. The method of claim 30 wherein said antimicrobial agent is a urinary anti-infective agent.

41. The method of claim 31 wherein said antifungal agent is Amphotericin.

42. The method of claim 31 wherein said antifungal agent is a pyrimidine derivative.

43. The method of claim 31 wherein said antifungal agent is a diazole or triazole-containing compound.

44. The method of claim 32 wherein said antiviral agent is a purine nucleoside or nucleotide.

45. The method of claim 32 wherein said antiviral agent is a pyrimidine nucleoside or nucleotide.

46. The method of claim 32 wherein said antiviral agent is an antiretroviral agent.

47. A method of reducing the toxicity of a first drug consisting of a penicillin, a cephalosporin, a macrolide, an epothilone, a quinolone, an aminoglycoside, an antituberculosis agent, or a urinary anti-infective agent, administered to a patient as therapy for an infectious disease, said method comprising administering an effective amount of the antimicrobial agent, or an antifungal agent, or an antiviral agent, and an effective amount of a formula I compound: 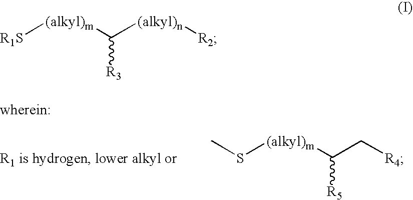
R₂ and R₄ are each individually SO₃⁻M⁺, PO₃²⁻M₂²⁺, or PO₂S²⁻M₂²⁺;
R₃ and R₅ are each individually hydrogen, hydroxy or sulfhydryl;
m and n are individually 0, 1, 2, 3 or 4, with the proviso that if m or n is 0, then R₃ is hydrogen; and
M is hydrogen or an alkali metal ion; or
a pharmaceutically acceptable salt thereof;
wherein the effective amount of the formula I compound is from four times by weight greater to five thousand times by weight greater than the amount of the antimicrobial agent, or the antifungal agent, or the antiviral agent administered.

48. The method of claim 47 wherein said formula I compound is administered to said patient at a time from fifteen minutes prior to thirty minutes prior to administration of the antimicrobial, antifungal or antiviral agent.

49. The method of claim 47 wherein said formula I compound is administered to said patient simultaneously with the antimicrobial, antifungal or antiviral agent.

50. The method of claim 47 wherein said formula I compound is administered to said patient intravenously.

51. The method of claim 47 wherein said formula I compound is administered to said patient orally.

52. The method of claim 47 wherein said antimicrobial, antifungal or antiviral agent is administered parenterally.

53. The method of claim 47 wherein said first drug is an antimicrobial agent.

54. The method of claim 47 wherein said first drug is an antifungal agent.

55. The method of claim 47 wherein said first drug is an antiviral agent.

56. The method of claim 53 wherein said antimicrobial agent is a penicillin.

57. The method of claim 53 wherein said antimicrobial agent is a cephalosporin.

58. The method of claim 53 wherein said antimicrobial agent is a macrolide.

59. The method of claim 58 wherein said antimicrobial agent is an epothilone.

60. The method of claim 53 wherein said antimicrobial agent is a quinolone.

61. The method of claim 53 wherein said antimicrobial agent is an aminoglycoside.

62. The method of claim 53 wherein said antimicrobial agent is an antituberculosis agent.

63. The method of claim 53 wherein said antimicrobial agent is a urinary anti-infective agent.

64. The method of claim 54 wherein said antifungal agent is Amphotericin.

65. The method of claim 54 wherein said antifungal agent is a pyrimidine derivative.

66. The method of claim 54 wherein said antifungal agent is a diazole or triazole-containing compound.

67. The method of claim 55 wherein said antiviral agent is a purine nucleoside or nucleotide.

68. The method of claim 55 wherein said antiviral agent is a pyrimidine nucleoside or nucleotide.

69. The method of claim 55 wherein said antiviral agent is an antiretroviral agent.
